(19) United States
(12) Patent Application Publication
(10) Pub. No.: US 2024/0025943 A1
(43) Pub. Date: Jan. 25, 2024

(54) METHOD FOR OBTAINING MATERIAL FROM PLANT CELL SURFACES

(71) Applicant: eleva GmbH, Freiburg (DE)

(72) Inventors: Berengar JAHN, Bruchsal (DE); HOLGER NIEDERKRÜGER, Malterdingen (DE); JONAS KOCH, Freiburg (DE); ANDREAS BUSCH, Ebringen (DE); ANDREAS SCHAAF, Freiburg (DE)

(73) Assignee: eleva GmbH, Freiburg (DE)

(21) Appl. No.: 17/769,570

(22) PCT Filed: Oct. 16, 2020
(86) PCT No.: PCT/EP2020/079143

§ 371 Date: Apr. 15, 2022

(30) Foreign Application Priority Data
Oct. 17, 2019 (EP) 19203842.0

(51) Int. Cl. C07K 1/14; C12N 9/40

(52) U.S. Cl. CPC C07K 1/145; C12N 9/2465; C12Y 302/01022

(57) ABSTRACT

The invention concerns a method for detaching expressed material from the surface or from the apoplast of plant cells, wherein the plant cells are treated with a rotor-stator in a liquid medium, wherein the specific heat from the rotor-stator introduced by rotation of the rotor is a maximum of 3 kJ per kg of the liquid medium and per g/L dry weight of the plant cells and the specific heat capacity introduced into the medium is a maximum of 1.5 kJ per kg of the liquid medium per minute and per g/L dry weight of the plant cells.

### FIELD OF THE INVENTION

The present invention relates to the isolation of proteins from cells.

### BACKGROUND OF THE INVENTION

Methods for isolating proteins from cells or cell complexes include osmotic lysis, enzymatic or chemical lysis, ultrasound treatment and mechanical digestion. As a rule, cells are comminuted with a homogenizer or mixer for the purposes of mechanical digestion.

Short treatment times can also be employed in order to divide cell complexes and in order to bring cells into suspension, whereupon only partial lysis of the total cell count occurs (Orellana-Escobedo et al., Plant Cell Rep. 2015, 34(3):425-33).

Whenever a protein is isolated from individual organelles or cell compartments, the cell organelles are usually isolated prior to digestion and then digestion is carried out using the isolate.

Witzel et al., Plant Methods 2011, 7:48; Leary et al., J Vis Exp. 2014; (94): 52113; and Córdoba-Pedregosa et al., Plant Physiology 1996, 112(3):1119-1125, describe methods for extracting proteins from the apoplast of plant cells. The space outside the protoplast is defined as the apoplast. It consists of the cell walls and the intercellular space. The methods described in those publications include an osmotic extraction with different infiltration solutions (for example salts) and centrifugation. That method, however, suffers from the disadvantage that in that extraction, only the materials that are made accessible by the infiltration can be extracted.

US 2015/0140644 A1 and AU 2017 202473 B2 describe methods for obtaining proteins from the apoplast with the steps of lysis of the cell wall, incubation and extraction. Enzymatic or chemical modifications to the proteins are possible therein.

One aim of the invention is to provide improved options for the isolation or extraction of the materials from the apoplast, in particular material secreted into the apoplasts.

### SUMMARY OF THE INVENTION

The present invention concerns a method for detaching expressed material from the surface or from the apoplast of plant cells, wherein the plant cells are treated with a rotor-stator in a liquid medium, wherein the specific heat from the rotor-stator introduced by rotation of the rotor is a maximum of 3 kJ per kg of the liquid medium and per g/L dry weight of the plant cells and the specific heat capacity introduced into the medium is a maximum of 1.5 kJ per kg of the liquid medium per minute and per g/L dry weight of the plant cells.

Similarly, in a further aspect, the invention concerns a method for detaching expressed material from the surface or from the apoplast of one or more plant cells, wherein the one or more plant cells is/are treated in a liquid medium with a rotor-stator, wherein the heat from the rotor-stator introduced by rotation of the rotor is a maximum of 30 kJ per kg of liquid medium and the heat capacity introduced into the medium is a maximum of 1.5 kJ per kg of liquid medium and per minute.

The parameters of both aspects may be combined, in particular because only reference values are involved and the spirit of the invention is served in both cases. All of the detailed descriptions of the invention and preferred embodiments described therein refer to all aspects of the invention.

### DETAILED DESCRIPTION OF THE INVENTION

The invention concerns a conservational rotor-stator treatment of plants or plant cells which, in contrast to the otherwise usually used homogenization, detaches material from the apoplasts of the cells or plants. In this regard, the protoplasts remain substantially intact and contamination of the expressed material to be isolated by cell components from the interior of the cell of protoplasts is avoided. In this regard, in accordance with the invention, the intensity and duration of the treatment with the rotor-stator is limited. It has been shown that by means of a rotor-stator treatment with low inputs of energy—determined as the specific heat or heat capacity—satisfactory detachment of the desired expressed material is possible. Excellent results could be obtained with a specific heat from the rotor-stator introduced by rotation of the rotor of a maximum of 3 kJ per kg of the liquid medium and per g/L dry weight of the plant cells as well as with a specific heat capacity into the medium introduced by rotation of the rotor of a maximum of 1.5 kJ per kg of the liquid medium per minute and per g/L dry weight of the plant cells. Equally good results are obtained when the heat from the rotor-stator introduced by rotation of the rotor is a maximum of 30 kJ per kg of the liquid medium as well as when the heat capacity introduced into the medium is a maximum of 1.5 kJ per kg of the liquid medium and per minute—the inventive concept is the same. In accordance with the invention, the plants or plant cells are not homogenized in this case, but only treated to the extent that the expressed material is detached from the surface or out of the apoplast.

With the method in accordance with the invention, expressed material is obtained from the surface of the cells or the apoplast (totality of cell walls and intercellular space). Material of this type usually reaches these sites by secretion and is also usually found in the culture medium for the plant cells. However, when developing the invention, it was established that large quantities of secreted material adheres to the surface, in particular to the cell wall or to the apoplasts. This adhering material is obtained in accordance with the invention, whereupon increases in production can be achieved by means of the method in accordance with the invention. Approximately ten-fold increases in yields compared with methods without a rotor-stator treatment, i.e. isolation of the secreted expressed material, were observed.

The treatment intensity, duration and capacity of the rotor-stator are selected so as to be within the maximum parameters in accordance with the invention in order to obtain a sufficient amount of the expressed material (desired product). However, the treatment intensity, duration and capacity, which represent the energy introduced, quantified as heat or specific heat, are limited, because product contamination occurs with higher inputs of energy.

Preferably, the specific heat from the rotor-stator introduced by rotation of the rotor is a maximum of 3 kJ per kg of the liquid medium and per g/L dry weight of the plant cells (abbreviated to 3 kJ/kg/(g/L) or 3 kJ/kg/g/L). Particularly preferably, the specific heat can be kept lower in order to further reduce all residual contamination. Thus, preferably, the specific heat introduced from the rotor-stator is a maximum of 2.75 kJ/kg/(g/L), or more preferably a maximum of 2.5 kJ/kg/(g/L), a maximum of 2.25 kJ/kg/(g/L), a maximum of 2 kJ/kg/(g/L), a maximum of 1.75 kJ/kg/(g/L), a maximum of 1.5 kJ/kg/(g/L), a maximum of 1.25 kJ/kg/(g/L), or a maximum of 1 kJ/kg/(g/L).

Again in accordance with the invention, the optional parameters of the introduced heat are independent of any reference to the plant quantity. In some embodiments this is of relevance because the rotor-stator delivers energy to the cell medium independently of the plant cells; this can manifest itself in heating up. Preferably, the heat from the rotor-stator introduced by rotation of the rotor is a maximum of 30 kJ per kg of the liquid medium (abbreviated to kJ/kg), preferably a maximum of 25 kJ/kg, a maximum of 20 kJ/kg, a maximum of 15 kJ/kg or a maximum of 10 kJ/kg.

This introduced specific heat or introduced heat can be adjusted, for example by means of temporally limited treatment periods and/or the intensity of the treatment (and by the same token, the parameters of specific heat capacity or heat capacity):

In the method in accordance with the invention, the rotor-stator is operated at a low intensity, for example at a low rotational speed. The heat (or heat capacity) of a specific method introduced at a specific intensity with selected parameters can be measured in a comparative experiment, for example by raising the temperature of water or another medium with a known heat capacity. When determining the heat of the method, other effects that influence the temperature, in particular heat losses, can be excluded or taken into account in the calculations in order to obtain the heat or heat capacity of the rotor-stator in this manner; preferably, the heat or heat capacity is determined in a Dewar flask.

Independently of the appliance, the introduced specific heat capacity or the introduced heat capacity is recognized as being relevant (as above, “specific” refers to the optional reference to the quantity of plant material). Preferably, the specific heat capacity introduced into the medium is a maximum of 1.5 kJ per kg of the liquid medium per minute and per g/L dry weight of the plant cells (abbreviated to kJ/kg/min/(g/L) or kJ/kg/min/g/L). Particularly preferably, this specific heat capacity is a maximum of 1.25 kJ/kg/min/(g/L), a maximum of 1 kJ/kg/min/(g/L), a maximum of 0.8 kJ/kg/min/(g/L), a maximum of 0.6 kJ/kg/min/(g/L), a maximum of 0.5 kJ/kg/min/(g/L), a maximum of 0.4 kJ/kg/min/(g/L), a maximum of 0.3 kJ/kg/min/(g/L), a maximum of 0.2 kJ/kg/min/(g/L), a maximum of 0.15 kJ/kg/min/(g/L), a maximum of 0.125 kJ/kg/min/(g/L), or a maximum of 0.1 kJ/kg/min/(g/L). In analogous manner to this, the heat capacity introduced into the medium is a maximum of 1.5 kJ per kg of the liquid medium and per minute (abbreviated to kJ/kg/min). Preferably, this heat capacity is a maximum of 1.25 kJ/kg/min, a maximum of 1 kJ/kg/min, a maximum of 0.8 kJ/kg/min, a maximum of 0.6 kJ/kg/min, a maximum of 0.5 kJ/kg/min, or a maximum of 0.4 kJ/kg/min.

The more intensive or lengthy the treatment, the higher is the quantity of material obtained. Preferably, the heat introduced by rotation of the rotor (of the rotor-stator) is at least 1 kJ per kg of the liquid medium, particularly preferably at least 2 kJ/kg, and/or the specific heat from the rotor-stator is at least 0.1 kJ per kg of the liquid medium and per g/L dry weight of the plant cells, particularly preferably at least 0.2 kJ/kg/(g/L).

Preferably, the heat capacity introduced into the medium by rotation of the rotor is at least 0.2 kJ per kg of the liquid medium and per minute, preferably at least 0.4 kJ/kg/min, and/or the specific heat capacity into the medium is at least 0.02 kJ per kg of the liquid medium per minute and per g/L dry weight of the plant cells, preferably at least 0.04 kJ/kg/min/(g/L).

The expressed material preferably contains proteins. Proteins, in particular recombinant expressed proteins, can be specifically directed down the secretion pathway with appropriate signal sequences and thus be steered towards concentration on the surface or in the apoplast. Preferably, the expressed material is in the apoplast of the plant cells, from which it can be obtained with the method in accordance with the invention. More preferably, the expressed material is secreted material, preferably proteins secreted through the cell membrane or cell wall.

In accordance with the invention, a rotor-stator is used in order to process the plant cells in the liquid medium to obtain the expressed material.

A rotor-stator comprises at least one rotor which applies shear forces to the plant cells because of its rotational motion. By means of the shear forces, the surface of the plant cells or the apoplast and the cell walls is (structurally) relaxed, mechanically influenced or rubbed off or partly removed from the surface.

The rotor rotates relative to a stator. The stator may be a housing sheath or a counterpart to the rotor. The rotor may have cutting or shear elements with edges or shear surfaces. A common construction has a comb structure, wherein a plurality of shear projections (for example teeth or tines) which are usually disposed parallel to the axis of rotation, exert shear or cutting actions. As an example, a “plurality” may be 2, 3, 4, 5, 6, 7, 8 or more shear projections.

The stator can be configured as a counterpart to the rotor and in particular its cutting or shear elements. Optionally, like the rotor, the stator may have its own cutting or shear elements, and for example also have a comb structure. Constructions of this type are known in rod homogenizers, as described in DE 10 2005 031 459 A1.

In other embodiments, the stator may be a housing structure, as is usual, for example, in flow-through homogenizers. An example of a flow-through rotor-stator is described in WO 2009/062610 A1.

Examples of rotor-stators are a rod homogenizer or a shear pump. Shear pumps are used for flow-through systems in particular.

Preferably, there is a gap, for example at least the size of one or more plant cells, between the rotor and stator so that plant cells at least in the form of protoplasts can pass between the rotor and stator. Suitable gap sizes are 50 μm, 70 μm, 80 μm, 100 μm, 150 μm or more as well as any region between these distances, preferably up to a maximum of 500 μm or up to 300 μm or up to 200 μm.

As noted, the intensity is kept low in order to conserve the plant cells in the form of protoplasts, to prevent or reduce contamination by cell interiors of the secreted expressed material which is to be harvested. In normal rotor-stator models, to this end, the rotational speed is reduced, for example in embodiments in which the rotor is operated with a maximum rotational speed of 15,000 revolutions per minute, preferably 1,000 to 15,000 revolutions per minute. Possible rotational speeds are 3,000 to 14,000, 4,000 to 13,000, 5,000 to 12,000 or 6,000 to 11,000 revolutions per minute.

The plant cells may be in a container into which the rotor-stator is introduced. To this end, the rotor-stator may be introduced into a container with the medium. This “batch” construction (for a discontinuous operation) is used with rod homogenizers in particular. On larger scales, flow-through rotor-stators are preferably used. In accordance with this embodiment, the rotor-stator may have an interior which has at least one inlet and outlet, by means of which the liquid medium is continuously fed through the interior. An example of this case is the shear pump for a continuous method.

Preferably, the stator delimits a volume of 10 cm³ (0.01 L) to 1 m³ (1000 L). Preferred volumes are 0.1 L to 800 L, or 0.5 L to 600 L or 1 L to 400 L, 2 L to 200 L. Volumes of up to 100 L are preferred, particularly preferably 0.65 L to 50 L, for example 1 L to 40 L. These volumes are particularly suitable for the treatment of culture media for plant cells.

Preferably, the quantity of the treated liquid medium is up to 50000 kg, preferably 0.5 g to 50,000 kg, for example 1 g to 25,000, 2 g to 10,000 kg, 5 g to 5,000 kg, 10 g to 2,500 kg, 20 g to 1,000 kg, 30 g to 500 kg, 50 g to 250 kg, 100 g to 100 kg, 200 g to 50 kg, 500 g to 250 kg, 1 kg to 100 kg, 2 kg to 50 kg or 4 kg to 20 kg. Quantities of this type are those preferably used per run in a discontinuous method.

Preferably, the plant cells are present in a concentration of 0.2 g/L to 60 g/L in the liquid medium (mass of plant cells as the dry weight). Preferred concentrations in this regard for the plant cells (always as the dry weight) are 0.5 g/L to 50 g/L, 1 g/L to 40 g/L, 2 g/L to 30 g/L, 4 g/L to 20 g/L, particularly preferably approximately 10 g/L, for example 5 g/L to 15 g/L. These plant cell concentrations are processed particularly efficiently with the rotor-stator.

Preferably, the plant cells are treated with the rotor-stator for 2 min to 150 min. In continuous methods, these times refer to the average treatment time for the plant cells.

Particular preferred times are 3 min to 120 min, 5 min to 100 min, 8 min to 80 min, 10 min to 60 min, or particularly preferably 12 min to 40 min. With large culture volumes, longer rotor-stator treatments may be undertaken. Preferred further possible times are 1 h to 24 h, preferably 2 h to 20 h, 3 h to 16 h, 4 h to 12 h. In this manner, all preferred treatment times are in the range 3 min to 24 h, and every range between the cited treatment times or in fact longer.

Preferably, the plant cells can be cultured in a suspension culture. This suspension can be processed directly as the liquid medium in the method in accordance with the invention. Alternatively, moss may also firstly be isolated, for example from a solid culture, liquid culture or suspension culture, and then suspended in an aqueous medium under conditions suitable for carrying out the rotor-stator treatment. Plants which are particularly suitable for the method in accordance with the invention are non-ligneous plants. Preferred plants are algae and mosses, in particular bryophytes. Preferably, the bryophyte plant or cell is a moss, preferably P. patens. The bryophyte may be any bryophyte, but is preferably selected from moss, liverwort or hornwort, particularly preferably from the Bryopsida class or the genuses Physcomitrella, Funaria, Sphagnum, Ceratodon, Marchantia and Sphaerocarpos. Physcomitrella patens is particularly preferred. Most preferably, the method in accordance with the invention is carried out using cells from plant tissue such as protonema from the liverwort Physcomitrella patens. Preferred algae are selected from green algae, for example from the Chlorellales order, preferably from the Chlorellaceae family, more preferably from the genus Auxenochlorella or Chlorella, in particular Chlorella vulgaris, and from the Volvocales order, preferably from the Haematococcaceae family, more preferably from the genus Haematococcus, in particular Haematococcus pluvialis and from the Eustigmatales order, preferably from the Loboceae, Chlorobothryaceae, Pseudocharaciopsidaceae and Eustigmataceae family. Further preferred plants are tobacco, beans or lentils. Preferably, the plant is a water plant, for example from the genuses Lemna, Spirodela, Landoltia, Wolffia or Wolffiella.

Plant cells constitute the subject matter of the invention. The term “plant cells” as used herein may refer to isolated cells, an individualized cell, but also to a cell in or from plant tissue, preferably a tissue selected from the callus, protonema, phloem, xylem, mesophyll, stem, leaves, thallus, chloronema, rhizoid or gametophore, or a cell in a plant organism.

In the method in accordance with the invention, the medium preferably has a physiological pH, in particular in order, as described above, to conserve the protoplasts (cell components inside the cell wall, in particular from the cell membrane), in order to prevent contamination of the expressed material in the apoplast/on the cell surface. The pH of the liquid medium is preferably between 3.5 and 8.5, particularly preferably between 4 and 8, or between 4.5 and 7, or between 5 and 6.5, in particular between 5.5 and 6, or combinations of these values, such as a pH of 5 to 8.

Again in order to conserve the protoplasts, the osmolarity of the liquid medium is preferably physiological, in particular in order to avoid swelling stress, for example when the osmolarity is too low. If appropriate, an upper limit to the osmolarity may be provided in order to avoid osmotic shrinking stress. Preferably, the medium has an osmolarity of at least 0.1 osmol/L, or preferably at least 0.150 osmol/L. The osmolarity may be adjusted by dissolved substances such as salts or other medium components for example sugar or sugar alcohol. Preferably, an alkali metal salt such as Na⁺ and/or K⁺ is provided.

Preferably, a halide such as Cl⁻ or F⁻ or I⁻, a phosphate or an acetate is provided as the anion. Buffer components such as Tris (tris(hydroxymethyl)aminomethane) are also possible.

Furthermore, in order to further conserve the protoplasts, surfactant polymers may be added to the liquid medium for the rotor-stator treatment. Surfactant polymers have, for example, been described in WO 2013/156504 A1 and preferably encompass uncharged polymers such as emulsifying agents, for example polyalkylglycols, in particular polyethyleneglycol. In particular, the polymer is a non-ionic water-soluble surfactant polymer. Preferably, it does not denature proteins. Examples are polymers or copolymers selected from polyethers such as polyalkylglycol, polysorbates or polyvinylpyrrolidone, polyvinylalcohol, water-soluble cellulose derivatives such as hydroxypropylcellulose, hydroxypropylmethylcellulose, carboxymethylcellulose or hydroxyethylcellulose, vinylpyrrolidone vinylacetate copolymer (copovidone), polyvinylacetate, partially hydrolysed polyvinylalcohol, polyvinylalcohol-polyethyleneglycol copolymers, and mixtures thereof. Further possibilities are polysorbate, for example polyoxyethylene sorbitan monolaurate, polyoxyethylene sorbitan monooleate, polyoxyethylene sorbitan monopalmitate, polyoxyethylene sorbitan monostearate, polyoxyethylene sorbitan tristearate, preferably polysorbate 80 (polyoxyethylene (20) sorbitan monooleate, Tween® 80), polyoxyethylene (40) stearate. The surfactant polymer is preferably present in the medium in a concentration of at least 0.05% by weight, particularly preferably at least 0.08%, at least 0.1% or at least 1.5% (all percentages as % by weight). The molecular weight of the surfactant polymer, for example PEG and the like, is preferably at least 500 Da, particularly preferably at least 1,000 Da, at least 1,500 Da, at least 2,000 Da, at least 3,000 Da, at least 4,000 Da, at least 6,000 Da, at least 8,000 Da, at least 10,000 Da, at least 20,000 Da, or at least 30,000 Da. Particularly preferably, the molecular weight is between 500 Da and 2,000,000 Da, preferably between 1,000 Da and 200,000 Da or between 1,200 Da and 80,000 Da.

The liquid medium is preferably aqueous, in particular water or water mixtures which are compatible with cells. In particular, it may be a culture medium for (and with) plant cells, insofar as the plants have not been separated from it in advance.

Preferably, the expressed material has been expressed prior to treating the plant cells with the rotor-stator, so that the plant cells collect on the surface or in the apoplast. In this regard, the plant cells may be cultured and/or allowed to grow, for example in a medium under growth conditions for plants (nutrient medium, light), as is generally known (see, for example Frank et al. Plant Biol 7, (2005):220-227). Expression or culture is preferably carried out for 13 min to 1 month (30 days) or longer, such as 2 months (60 days), for example 1 h to 22 days, or 5 h to 15 days, for example 10 h to 7 days or 20 h to 3 days. In a continuous cell culture with regular removal of cells in order to obtain the product (expressed material), these time ranges or minimum times may correspond to a mean period for a cell under culture.

Other methods which damage the protoplasts or lyse or homogenize them should be avoided. The cell wall is preferably not lysed, in particular not lysed enzymatically and/or not chemically and/or not osmolytically and/or not using ultrasound. Preferably—apart from the rotor-stator treatment in accordance with the invention—the cell wall should remain untouched or intact. In particular, the cell membrane (protoplast) should remain intact, whereupon in the method in accordance with the invention, the vitality of the cells does not play any particular role, however contamination of the liquid medium by components of the cell interior, in particular the cell plasma, should be avoided.

The present invention will now be described in more detail by means of the figures and examples below, without limiting the invention to these embodiments.

### FIGURES

FIG. 1: Determination of energy input in water by the T25 Ultra Turrax rod homogenizer (IKA/Staufen). (A) temperature profile in 1.5 L water at a rotational speed of 10,000 rpm. (B) Calculated energy input [KJ]. (C) Calculated energy input [KJ/kg].

FIG. 2: Determination of energy input in water using the FSP712VC-2.2 kW-FU shear pump homogenizer (Fristram/Hamburg). (A) temperature profile in 50 L water at a rotational speed of 2,800 rpm. (B) Calculated energy input [KJ]. (C) Calculated energy input [KJ/kg].

FIG. 3: Percentage release of biomass-bound product (moss-aGal) during treatment with the shear pump (broken line) and T25 Ultra Turrax rod (solid line). The shear pump treatment of the reactor culture was carried out in a volume of 50 L. The treatment of the reactor culture with the T25 Turrax rod was carried out in a volume of 0.65 L.

FIG. 4: Specific energy input using the T25 Ultra Turrax rod (A) and shear pump (B), both for 10 g/L dry biomass. Specific energy input in accordance with comparative example T25 Ultra Turrax rod at 19,000 rpm with 1 g/L dry biomass (C).

FIG. 5: Percentage release of biomass-bound product (moss-aGal) during treatment with the shear pump (broken line) and T25 Ultra Turrax rod (solid line). The shear pump treatment of the reactor culture was carried out in a volume of 50 L. The treatment of the reactor culture with the T25 Turrax rod was carried out in a volume of 0.65 L. Biomasses: 9.2 g/L (shear pump), 8.2 g/L (T25).

FIG. 6: Western Blot analogous for product release (moss-aGal) compared with release of intracellular marker proteins (Rubisco, large subunit). Intracellular proteins in salt-containing media (for example 20 mM Tris, 100 mM NaCl, pH=7) for inputs of energy up to 32.9 KJ/kg detectable in minimum quantities. Under osmotic stress conditions (demineralized water), detectable from approximately 10 KJ/kg. Target protein quantities (moss-aGal) increase in salt-containing media and under osmotic stress conditions as a function of the input energy.

FIG. 7: Microscopic analysis of the T25 process in demineralized H₂O. Up to an energy input of 16.5 kJ/kg, the moss cells retain their integrity. At an energy input of 32.9 KJ/kg, the integrity of the cells is there, but more particles are clearly present, which is an indication of the beginning of cell digestion.

FIG. 8: Microscopic analysis of the T25 process in salt-containing buffer (20 mM Tris, 100 mM NaCl, pH=7). Up to an energy input of 16.5 kJ/kg, the moss cells retain their integrity. At an energy input of 32.9 KJ/kg, the integrity of the cells is there, but more particles are clearly present, which is an indication of the beginning of cell digestion.

FIG. 9: Microscopic analysis of the shear pump process in salt-containing medium (reactor culture). Up to an energy input of 18.41 kJ/kg, the moss cells retain their integrity. Beyond an energy input of 27.20 KJ/kg, the integrity of the cells is there, but more particles are clearly present, which is an indication of the beginning of cell digestion.

FIG. 10: Microscopic analysis of the shear pump process in demineralized H₂O. Up to an energy input of 9.62 kJ/kg the moss cells retain their integrity. Beyond an energy input of 18.41 KJ/kg, the integrity of the cells is there, but more particles are clearly present, which is an indication of the beginning of cell digestion.

FIG. 11: Microscopic analysis of moss cells in an ultrasound process as a comparative image for cell digestion. After just a brief period (1 min of ultrasound treatment), the cells lose their integrity. After just 3 min, only cell fragments and empty cell debris can be seen (100% in 50 mL sample).

FIG. 12: Comparative example: Determination of energy input in water using the T25 homogenization tool (IKA/Staufen) at high energy, a rotational speed of 19,000 rpm. (A) temperature profile in 1 L water. (B) Calculated energy input [KJ]. (C) Calculated energy input [KJ/kg].

FIG. 13: Comparative example: Significantly faster product release at 19,000 rpm. In particular up to an energy input of 7 KJ/kg. Beyond an energy input of 84 KJ/kg, loss of product due to high temperatures and shear stress.

### EXAMPLES

### Example 1: Determination of Energy Input Using Turrax Rod and Shear Pump

The parameter used for the energy input in aqueous media was the temperature as a measurable variable. For the T25-S25N-18G Turrax rod (IKA Staufen), 1.5 L of H₂O was dispersed for 30 min at 10,000 rpm in a Dewar flask and the temperature profile was measured. The room temperature during the experiment was between 20.5 and 20.7° C. Energy input data was calculated using the specific heat capacity (4,190 J kg⁻¹ K⁻¹) of H₂O. Energy input data which could be compared with the literature (Orellana-Escobedo et al., Plant cell Rep. 2015, 34(3), 425-433) were also obtained at 19,000 rpm.

For the shear pump (shear pump FSP 712, Fristam Hamburg), 50 L of H₂O was circulated at 2,800 rpm from a Nalgene container using reinforced PVC tubing. The temperature measurement in the Nalgene container was carried out using a temperature sensor (G002.1 precision thermometer, Carl Roth). The experiment was set up in a temperature-controlled chamber at 19° C. Heat losses into the environment were ignored in this experimental setup. Energy input data was calculated using the specific heat capacity (4, 190 J·kg⁻¹ K⁻¹).

### Example 2: Production of a Moss Culture

Axenic culture of moss production strains was carried out for 3-4 weeks in 200 L Single Use bioreactor bags (Cellbag 200, GE Healthcare) on Wave™ Rocking Motion bioreactors (Wave200, Ge Healthcare). The culture parameters were a shaking frequency of 19 to 25 rpm, shaking angle of 90, temperatures of 24-26° C. and a gas flow of 2 L/min and enrichment of the air supply with 2% CO₂. Illumination was with 4 LED modules installed above the bioreactor bag (reference number 120268 to 120282, Infors AG) with “warm white” LEDs. Moss culture was carried out under 24 h illumination. SM07 (100 mM NaCl, 6.6 mM KCl, 2.0 mM MgSO₄×7H₂O, 1.8 mM KH₂PO₄, 20.4 mM Ca(NO₃)₂×4H₂O, 0.05 mM Fe Na-EDTA, 4.9 mM MES, 0.1% (w/v) PEG4000, 100.26 μM H₃BO₃, 0.11 μM CoCl₂×6H₂O, 0.1 μM CuSO₄×5H₂O, 5 μM KI, 85.39 μM MnCl₂×4H₂O, 1.03 μM Na₂MoO₄×2H₂O, 0.11 mM NiCl₂×6H₂O, 0.04 Na₂SeO₃×5H₂O, 0.039 Zn-acetate×2H₂O) supplemented with 1000× Nitsch vitamins (Nitsch vitamin mixture, Duchefa, see manufacturer's specifications) was used as the mineral salt medium. The pH of 5-6 was set using a WAVEPOD I and Pump20 (GE Healthcare) to automatically add 0.25 M H₂SO₄ and 0.25 M NaOH. Recombinant α-galactosidase (αGal or α-Gal A) was expressed as described in WO 2016/146760 A1 (“moss-αGal”).

### Example 3: Release of Moss-Bound Product and Analytical Methods

In order to analyse the temporal profile for the release of moss-bound product, the culture obtained from Example 2 was exposed to the T25-S25N-18G Turrax rod and also to the shear pump FSP 712 at different inputs of energy. c_{PL} product concentration determinations (c_{PL}) for released product were carried out using moss-aGal ELISA (Biogenes/Germany). Detection of the degree of cell digestion was carried out using microscopic image analysis of the moss cells (microscope: Axiovert 200 oper Stemi SV11 with AxioCam camera, AxioSoft software and KL 1500 LCD cold light source (Carl Zeiss). A comparison with microscopic images for total digestion was possible by means of microscopic analyses of a 50 mL ultrasound digestion (probe: UW2070, Bandelin; amplifier: HD 2070, Bandelin) with 100% power for 20 min. On a molecular level, qualitative analysis was carried out using Western Blot on the released product as well as using the intracellular marker protein Rubisco. The primary antibodies used were anti-aGal (H00002717-D01P, abnova) and anti-Rubisco (AS03037, Agrisera) as well as anti-rabbit HRP (abcam, AS03037) as the secondary antibodies.

To analyse the relationship between released (c_{PL}) and releasable moss-bound product (c_{PX}), the untreated culture underwent cell digestion using a ball mill (steel balls: RB-3/G20W, Schleer; ball mill: MM300, Retsch). After separating the cell debris, the product concentration was determined using ELISA (Biogenes). In this regard, aGal (α-galactosidase, also known as “moss-aGal”), a protein expressed into the apoplastic void, was assayed.

### Example 4: Results and Discussion

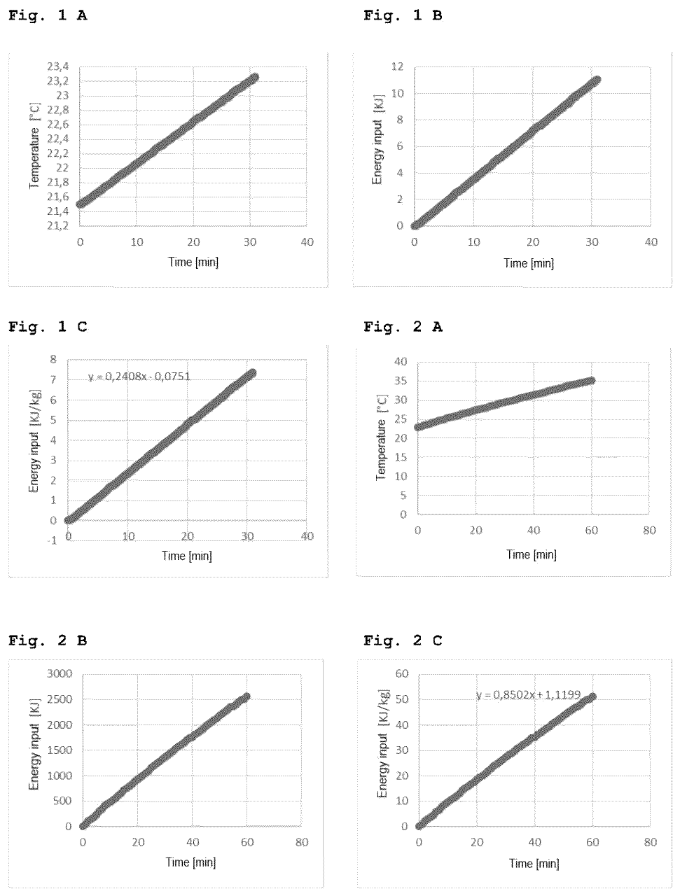

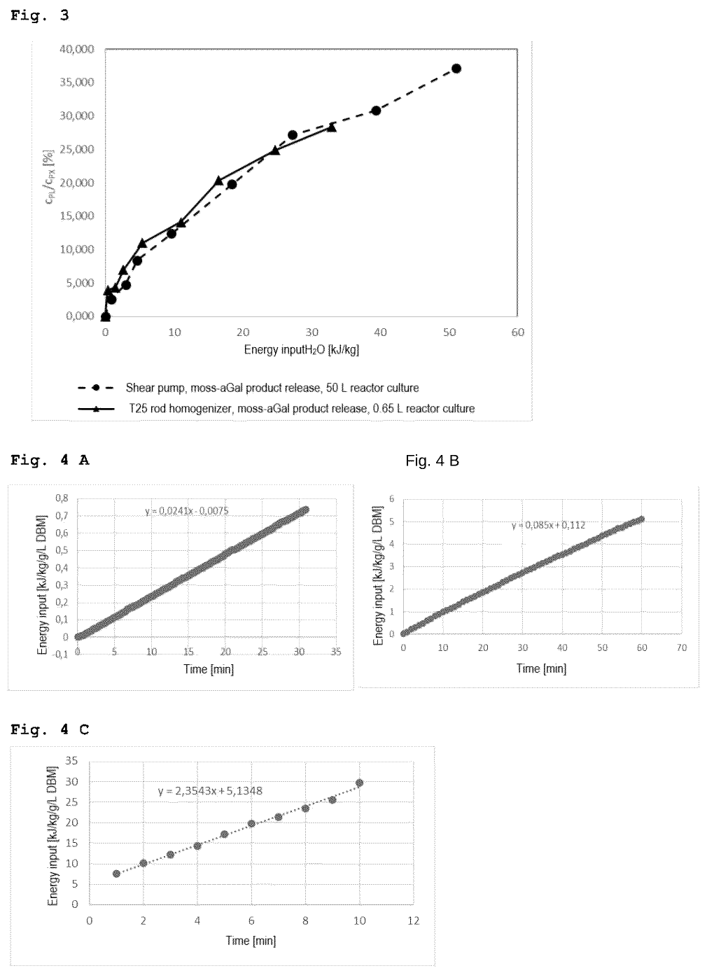

The inputs of energy (as heat, in kJ/kg, kg with respect to the liquid medium) for two different homogenizers—a T25-S25N-18G Turrax rod as well as a FSP 712 shear pump—were established in a culture medium (kg) by means of temperature measurements (FIGS. 1-5). In this regard, the homogenizer was set to a low rotational speed so that a low heat capacity (in kJ/kg/min) was produced. The total heat over a specific period of time (0-60 min) was determined in this manner. In this regard, the energy input in water was determined using the specific heat coefficient of H₂O [4.182 KJ/kg*K] based on the measured temperature. In this regard, the heat (FIGS. 1-3) or the specific heat with respect to the dry weight of the plant part (FIGS. 4-5) was calculated.

FIGS. 3 and 5 show the product release of the desired protein (recombinant aGal from moss, “moss-aGal”), which are deposited on the surface or in the apoplast. The release increases with increasing treatment.

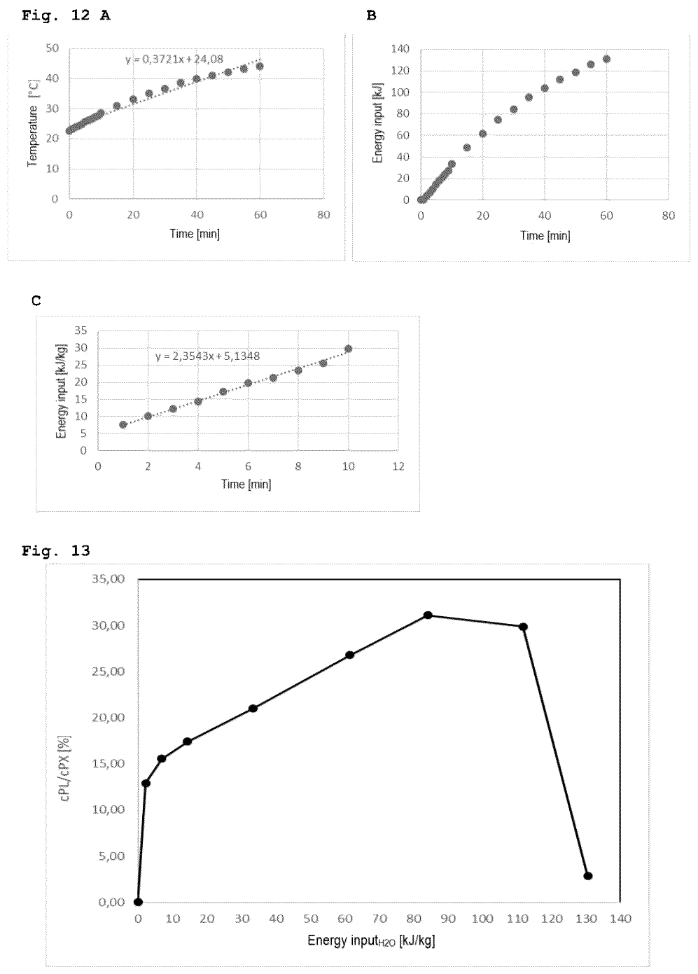

In comparative experiments, the Turrax rod was operated at a higher heat capacity and in fact at 19,000 rpm (FIGS. 4C, 12-13). The heat capacity was approximately 100 times higher than with the conservational treatment at 10,000 rpm (compare FIGS. 4A and 4C). The operation at a higher heat capacity rapidly led to product release, but also to destruction of the cells (protoplasts), so that extracellular products were contaminated with components of the interior of the cell. These effects were already occurring after 1 minute.

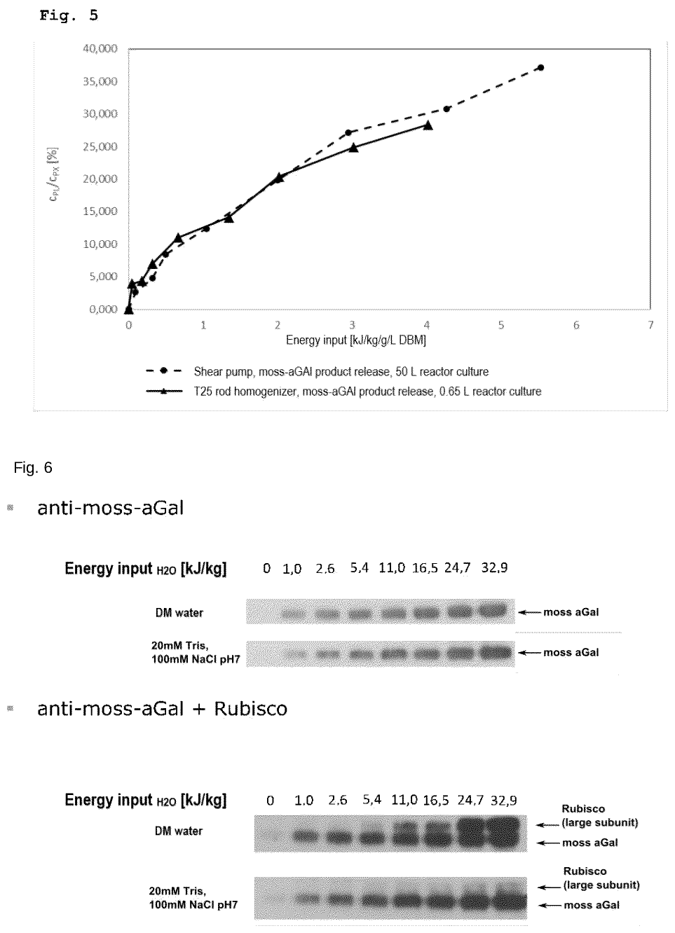

FIG. 6 shows the quality of the release of product from the extracellular protein (moss aGal) and from the intracellular protein Rubisco. Rubisco is present in plant cells in high concentrations and was therefore considered to be a highly sensitive marker for the escape of cell contents. In the experiments in a physiological salt solution, at higher inputs of energy (heat) of more than 32.9 kJ/kg, an increasing release of Rubisco was observed. In a comparative experiment with demineralized water (DM water), the contamination with Rubisco occurred even sooner, from approximately 10 kJ/kg, because osmolytic effects were occurring in addition to the shear stress from the homogenizer. The different heats (inputs of energy) were controlled using the treatment time.

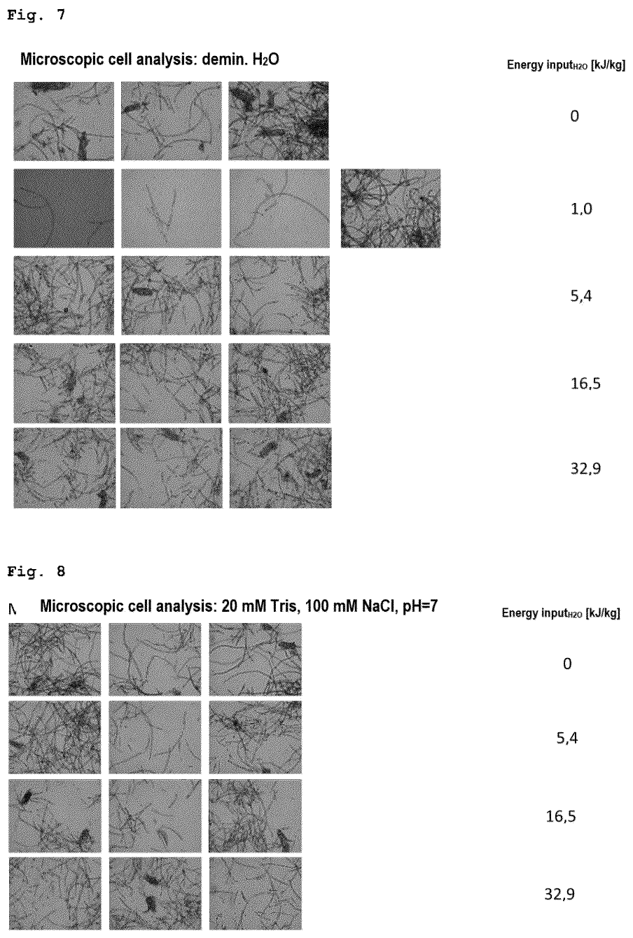

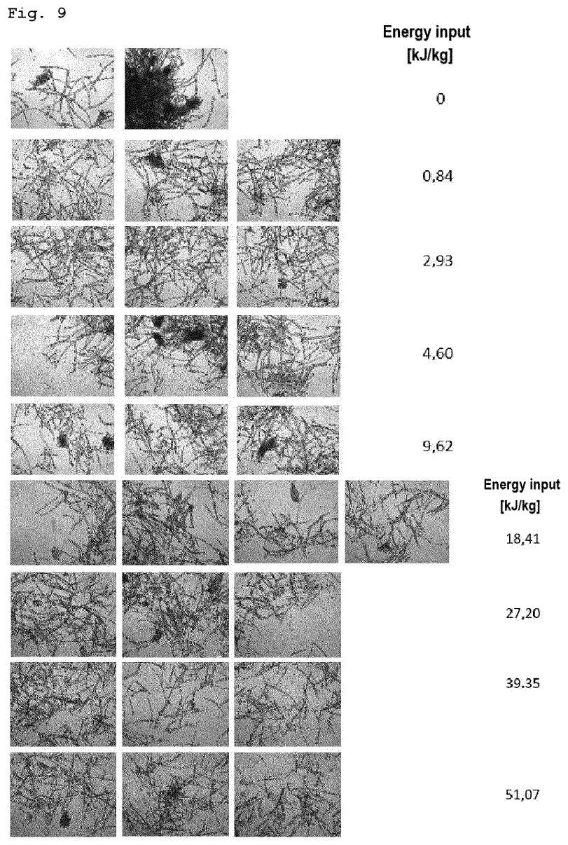

The microscopic analysis of the cell complexes following treatment reflects these results. FIG. 7 shows the results after treatment with the Ultraturrax rotor-stator in demineralized water; FIG. 8 after treatment with the Ultraturrax rotor-stator under physiological conditions (20 mM Tris, 100 mM NaCl, pH=7). FIG. 9 shows the cell complexes after treatment with the shear pump under physiological conditions. With increasing treatment time (heat), the number of particles, presumably formed by destroyed cells, increased. From approximately 30 kJ/kg, increased cell digestion arose. By way of comparison, FIG. 10 shows the experiments in demineralized water with the shear pump. Here, comparable particles appeared at approximately 20 kJ/kg.

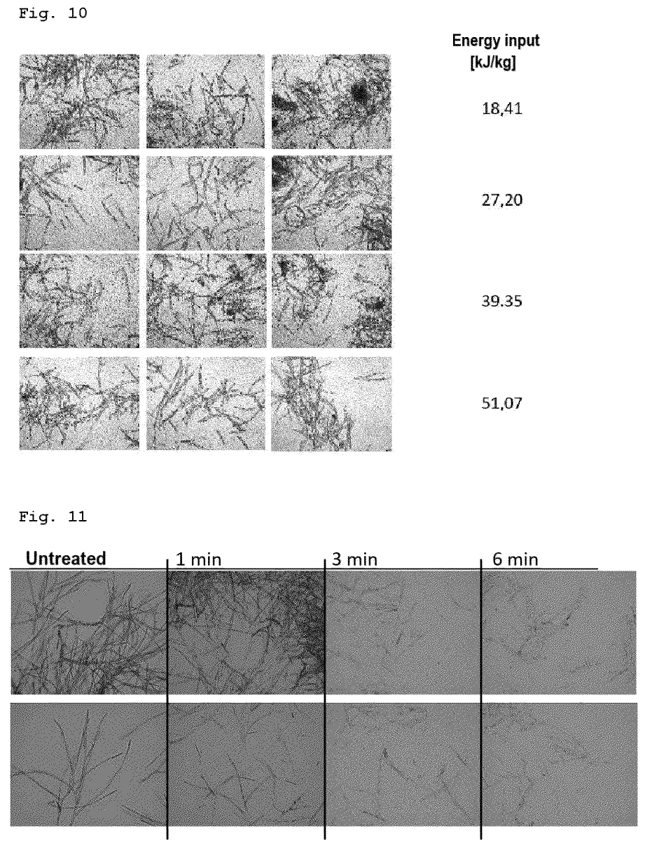

By way of comparison with FIGS. 7-10, FIG. 11 shows cell digestion using ultrasound. The cells have lost their integrity after just 1 min.

This shows that on the one hand, the energy input per unit of time (intensity of rotation of the rotor-stator; heat capacity) should be limited, and on the other hand that the absolute energy input (heat)—in the experiment here is controlled by the treatment time. This parameter may be included as is, or is related to the biomass (dry biomass, TBM). Appropriate figures for a conservational method, which releases as many as possible of the absorbed or apoplast-bound products and in doing so keeps the protoplasts largely intact, are a maximum of 3 kJ/kg per g/L dry weight and a maximum of 1.5 kJ/kg/min per g/L dry weight or a maximum of 30 kJ/kg and a maximum of 1.5 kJ/kg/min. Possible treatment times with low heat capacities of this type are 2 min to 150 min—depending on the intensity of rotation, as a rule longer than the short but intensive treatments used until now.

1. A method for detaching expressed material from the surface or from the apoplast of plant cells, wherein the plant cells are treated with a rotor-stator in a liquid medium, wherein the specific heat from the rotor-stator introduced by rotation of the rotor is a maximum of 3 kJ per kg of the liquid medium and per g/L dry weight of the plant cells and the specific heat capacity introduced into the medium is a maximum of 1.5 kJ per kg of the liquid medium per minute and per g/L dry weight of the plant cells.

2. The method as claimed in claim 1, characterized in that the expressed material is in the apoplast of the plant cells.

3. The method as claimed in claim 1, characterized in that the heat from the rotor-stator introduced by rotation of the rotor is at least 1 kJ per kg of the liquid medium, and/or the specific heat from the rotor-stator is at least 0.1 kJ per kg of the liquid medium and per g/L of dry weight of the plant cells.

4. The method as claimed in claim 1, characterized in that the heat capacity introduced into the medium by rotation of the rotor is at least 0.2 kJ per kg of the liquid medium and per minute, and/or the specific heat capacity into the medium is at least 0.02 kJ per kg of the liquid medium per minute and per g/L of dry weight of the plant cells.

5. The method as claimed in claim 1, characterized in that the expressed material contains proteins, and/or in that the expressed material is secreted material, preferably proteins secreted through the cell membrane.

6. The method as claimed in claim 1, characterized in that the rotor-stator is introduced into a container with the medium.

7. The method as claimed in claim 1, characterized in that the rotor-stator has an interior which has at least one inlet and outlet via which the liquid medium is continuously fed through the interior.

8. The method as claimed in claim 1, characterized in that the stator delimits a volume of 10 cm³ to 1 m³, and/or in that the quantity of the treated liquid medium is up to 50 kg, preferably 0.5 g to 50 kg.

9. The method as claimed in claim 1, characterized in that the plant cells are in a concentration of 0.2 g/L to 60 g/L in the liquid medium (mass of plant cells as dry weight).

10. The method as claimed in claim 1, characterized in that the plant cells are moss cells, preferably P. patens cells.

11. The method as claimed in claim 1, characterized in that the rotor is operated at a maximum rotational speed of 15,000 revolutions per minute, preferably 1,000 to 15,000 revolutions per minute.

12. The method as claimed in claim 1, characterized in that the rotor-stator is a rod homogenizer or a shear pump, and/or wherein the stator has a comb structure.

13. The method as claimed in claim 1, characterized in that the heat from the rotor-stator introduced by rotation of the rotor is a maximum of 30 kJ per kg of the liquid medium and the heat capacity introduced into the medium is a maximum of 1.5 kJ per kg of the liquid medium and per minute.

14. The method as claimed in claim 1, characterized in that the medium has a pH in the range 5 to 8 and/or an osmolarity of at least 0.1 osmol/L.

15. The method as claimed in claim 1, characterized in that the plant cells are treated with the rotor-stator for 2 min to 150 min.

16. The method as claimed in claim 2, characterized in that the heat from the rotor-stator introduced by rotation of the rotor is at least 1 kJ per kg of the liquid medium, and/or the specific heat from the rotor-stator is at least 0.1 kJ per kg of the liquid medium and per g/L of dry weight of the plant cells.

17. The method as claimed in claim 2, characterized in that the heat capacity introduced into the medium by rotation of the rotor is at least 0.2 kJ per kg of the liquid medium and per minute, and/or the specific heat capacity into the medium is at least 0.02 Id per kg of the liquid medium per minute and per g/L of dry weight of the plant cells.

18. The method as claimed in claim 3, characterized in that the heat capacity introduced into the medium by rotation of the rotor is at least 0.2 Id per kg of the liquid medium and per minute, and/or the specific heat capacity into the medium is at least 0.02 kJ per kg of the liquid medium per minute and per g/L of dry weight of the plant cells.

19. The method as claimed in claim 2, characterized in that the expressed material contains proteins, and/or in that the expressed material is secreted material, preferably proteins secreted through the cell membrane.

20. The method as claimed in claim 3, characterized in that the expressed material contains proteins, and/or in that the expressed material is secreted material, preferably proteins secreted through the cell membrane.
